(19) United States
(12) Patent Application Publication
(10) Pub. No.: US 2026/0240532 A1
(43) Pub. Date: Aug. 20, 2026

(54) LEFT ATRIAL APPENDAGE OCCLUSION DEVICE

(71) Applicant: Elevate Innovations, LLC, Warren, NJ (US)

(72) Inventors: Jin PARK, Morris Plains, NJ (US); Daniel OLSEN, Asbury, NJ (US); Cory ZEGEL, Hoboken, NJ (US)

(21) Appl. No.: 19/545,432

(22) Filed: Feb. 20, 2026

Related U.S. Application Data
(60) Provisional application No. 63/760,940, filed on Feb. 20, 2025.

(51) Int. Cl. A61B 17/00; A61B 90/00

(52) U.S. Cl. CPC A61B 17/0057; A61B 90/39; A61B 2017/00477; A61B 2017/00579; A61B 2017/00592; A61B 2017/00597; A61B 2017/0061; A61B 2017/00623; A61B 2017/00632; A61B 2017/00646; A61B 2017/00862; A61B 2017/00867; A61B 2017/00951; A61B 2090/3966

(57) ABSTRACT

The present invention pertains to a left arterial appendage device comprising a cylindrical proximal hub and a plurality of looping struts forming a circle. The plurality of looping struts are interconnected only at the proximal hub, and are otherwise unconstrained to support a rolling action which allows the device to be delivered intravascularly through a catheter or similar introducer tube. The distal end of the looping strut moves radially inward towards the center of a spiral created by the looping strut, minimizing risk of causing damage to surrounding tissue, increasing density at the neck, and providing radial force for device retention.

### CROSS-REFERENCE TO RELATED APPLICATION

This application claims the benefit of U.S. Provisional Application No. 63/760,940, filed February 20, 2025, the entire disclosure of this application is hereby incorporated herein by reference.

### TECHNICAL FIELD

The present invention is generally related to the field of intravascular occlusion and more particularly to occlusion or closure of the left atrial appendage of the heart and the devices, delivery systems, and methods related to such a treatment.

### BACKGROUND

Left atrial appendage (LAA) occlusion or closure is a treatment option for patients with atrial fibrillation that are at risk for strokes due to blood clot formation. Typically, stroke risk mitigation is conducted through the usage of anticoagulants. However, some patients may be incapable of staying on anticoagulants long term, and as such require a separate treatment mechanism. The LAA is known to generate a large number of blood clots in patients with atrial fibrillation and, therefore, the risk should be addressed to minimize the risk of strokes.

LAA occlusion devices (hereinafter also referred to as "LAAD", "LAADs" "device", or "devices") function by introducing a physical substance in the neck of the LAA, thereby increasing the resistance to blood flow through the LAA. With sufficient resistance, the LAA embolizes, completely preventing blood flow through the LAA, and thereby removing the risk of emboli formation from the LAA. LAA occlusion devices are intravascularly delivered permanent implants intended to be a long-term treatment option for patients with high stroke risk.

Current technology involves deployment of a metallic mesh into the LAA opening, occluding the neck with the intent of embolizing the LAA to minimize risk of blood clot formation. For a LAAD to be successful, it must be capable of being accurately positioned in the LAA opening and subsequently maintaining the position post deployment. The LAAD must also be capable of providing significant resistance to blood flow, such that it promotes embolization of the LAA. In most cases, LAADs are formed of a shape memory alloy such as nitinol and are delivered intravascularly. In order to be capable of intravascular delivery, the LAA occlusion device must be loaded onto a delivery system and include a mechanism for deployment and detachment of the LAA occlusion device from the delivery system.

Current technology introduces significant risks regarding LAA occlusions. Specifically, the LAA occlusion device is at risk of migration post deployment, causing leaking and thereby limiting or negating the effectiveness of the LAAD and promoting blood clot formation. Additionally, the LAA occlusion device may generate trauma to the surrounding tissue during advancement or deployment of the device into the LAA, further increasing patient risk.

Therefore, there is a need for an LAA occlusion device that is capable of minimizing the surgical risks present in current technology, specifically around increasing occlusion effectiveness and minimizing trauma risk during implantation.

### SUMMARY

At least the above-discussed need is addressed and technical solutions are achieved in the art by various embodiments of the present invention. Some embodiments of the present invention pertain to a left atrial appendage (LAA) occlusion device (LAAD) comprising a network of metallic struts formed such that in the deployed configuration they create a looping pattern with a geometry that is capable of conforming to the LAA neck. In the undeployed configuration, the metallic struts of the LAA occlusion device are substantially linear with the metallic struts parallel to the axis of deployment. The LAA occlusion device transitions from the undeployed configuration to the deployed configuration through an unraveling action, providing an atraumatic method of advancement and deployment into the LAA. The LAA occlusion device may be loaded onto a push wire or similar delivery system capable of traversing the intravascular anatomy to allow the LAA occlusion device to reach and be deployed into the LAA. The LAA occlusion device may include protrusions along the metallic struts to aid in retention and minimize migration post deployment. The LAA occlusion device may incorporate a thin membrane that stretches between the metallic struts to facilitate occlusion of the LAA.

In some embodiments of the invention, the metallic struts are configured to increase resistance to blood flow through the LAA and increase the rate of embolization and closure.

In some embodiments of the invention, the metallic struts create a spiral pattern when viewed in a cross-section view.

In some embodiments of the invention, the metallic struts create a substantially circular pattern when viewed from a view perpendicular to the plane formed by the LAA neck.

In some embodiments of the invention, the metallic struts create a cylindrical shaped device when viewed in three dimensions.

In some embodiments of the invention, the LAA occlusion device in the deployed configuration has a length substantially less than in the undeployed configuration.

In some embodiments of the invention, the LAA occlusion device incorporates protrusions along the external metallic struts to facilitate adhesion to the LAA wall.

In some embodiments of the invention, the LAA occlusion device includes a proximal base to improve adhesion and minimize the risk of migration.

In some embodiments of the invention, the LAA occlusion device further includes a mechanism for providing radiopacity, including radiopaque markers, coatings, or integration of radiopaque materials into the device frame.

These and other embodiments of the invention are discussed in detail below.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 depicts a side view of the left atrial appendage device (LAAD) in the deployed configuration, according to an embodiment of the invention.

FIG. 2 depicts a side view of the left atrial appendage device in a partially deployed configuration, according to an embodiment of the invention.

FIG. 3 depicts a side view of the left atrial appendage device in a partially deployed configuration which is less deployed than the device in FIG. 2, according to an embodiment of the invention.

FIG. 4 depicts a side view of the left atrial appendage device in a partially deployed configuration which is less deployed than the device in FIG. 3, according to an embodiment of the invention.

FIG. 5 depicts a side view of the left atrial appendage device in an undeployed configuration, according to an embodiment of the invention.

FIG. 6 depicts an alternative side view of the left atrial appendage device in the deployed configuration, according to an embodiment of the invention.

FIG. 7 depicts an isometric view of the top of the left atrial appendage device from FIG. 1 in the deployed configuration, according to an embodiment of the invention.

FIG. 8 depicts an isometric view of the bottom of the left atrial appendage device from FIG. 1 in the deployed configuration, according to an embodiment of the invention.

FIG. 9 depicts a bottom view of the left atrial appendage device from FIG. 1 in the deployed configuration, according to an embodiment of the invention.

FIG. 10 depicts a bottom view of the left atrial appendage device in a partially deployed configuration similar to FIG. 2, according to an embodiment of the invention.

FIG. 11 depicts a bottom view of the left atrial appendage device in a partially deployed configuration similar to FIG. 4, according to an embodiment of the invention.

FIG. 12 depicts a bottom view of the left atrial appendage device in a deployed configuration where the body of the device is comprised of eight looping struts, according to an embodiment of the invention.

FIG. 13 depicts a bottom view of the left atrial appendage device in a deployed configuration where the body of the device is comprised of 12 looping struts, according to an embodiment of the invention.

FIG. 14 depicts a bottom view of the left atrial appendage device in a deployed configuration where the body of the device is comprised of 16 looping struts, according to an embodiment of the invention.

FIG. 15 depicts a singular looping pattern of the left atrial appendage device in the deployed configuration, according to an embodiment of the invention.

FIG. 16 depicts a singular looping pattern of the left atrial appendage device in a partially deployed configuration, according to an embodiment of the invention.,

FIG. 17 depicts a singular looping pattern of the left atrial appendage device in a partially deployed configuration which is less deployed than the device in FIG. 12, according to an embodiment of the invention.

FIG. 18 depicts a singular looping pattern of the left atrial appendage device in a partially deployed configuration which is less deployed than the device in FIG. 13, according to an embodiment of the invention.

FIG. 19 depicts an alternative singular looping pattern with less loops in the spiral compared to FIG. 15, according to an embodiment of the invention.

FIG. 20 depicts an alternative singular looping pattern with a more loops in the spiral compared to FIG. 15, according to an embodiment of the invention.

FIG. 21 depicts an alternative singular looping pattern with a smaller pitch compared to FIG. 15, according to an embodiment of the invention.

FIG. 22 depicts a looping strut with a barb on the outer loop in the deployed configuration, according to an embodiment of the invention.

FIG. 23 depicts a top isometric view of a left atrial appendage device which includes protruding barbs on the outer loop of the looping struts, according to an embodiment of the invention.

FIG. 24 depicts a side view of a barb or protrusion on the exterior edge of the outermost loop of the implant. In this embodiment, the barb or protrusion is of a width substantially equivalent to the width of the device struts, according to an embodiment of the invention.

FIG. 25 depicts a 2D flat view of an alternative embodiment of the barb or protrusion where it is incorporated into the width of the looping strut, according to an embodiment of the invention.

FIG. 26 depicts a 3D view of the alternative embodiment of the barb or protrusion depicted in FIG. 25, where the looping strut is curved while the barb or protrusion remains tangent to the curvature, according to an embodiment of the invention.

FIG. 27A depicts an isometric view of a left atrial appendage device with a proximal base comprised of a series of repeating struts, with a series of looping struts nested on top of the proximal base, according to an embodiment of the invention.

FIG. 27B depicts a bottom view of a left atrial appendage device with a proximal base comprised of a series of repeating struts, with a series of looping struts nested on top of the proximal base, according to an embodiment of the invention.

FIG. 28 depicts a sectional view of the left atrial appendage device depicted in FIG. 27B, where only the proximal base is visible, according to an embodiment of the invention.

FIG. 29 depicts an alternative version of the proximal base, comprising a larger number of struts arranged in a circular pattern around a central axis, according to an embodiment of the invention.

FIG. 30 depicts a side view of a left atrial appendage device in the deployed configuration with a proximal base comprised of a series of looping struts which form a base cavity, the proximal base sitting below or proximal to the deployed left atrial appendage device, according to an embodiment of the invention.

FIG. 31 depicts a bottom view of the left atrial appendage device depicted in FIG. 30, according to an embodiment of the invention.

### DETAILED DESCRIPTION

In the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘including at least A’ includes A as well as the possibility of one or more other additional elements besides A. In the same manner, the phrase, ‘including A’ includes A, as well as the possibility of one or more other additional elements besides A. However, the phrase, ‘including only A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

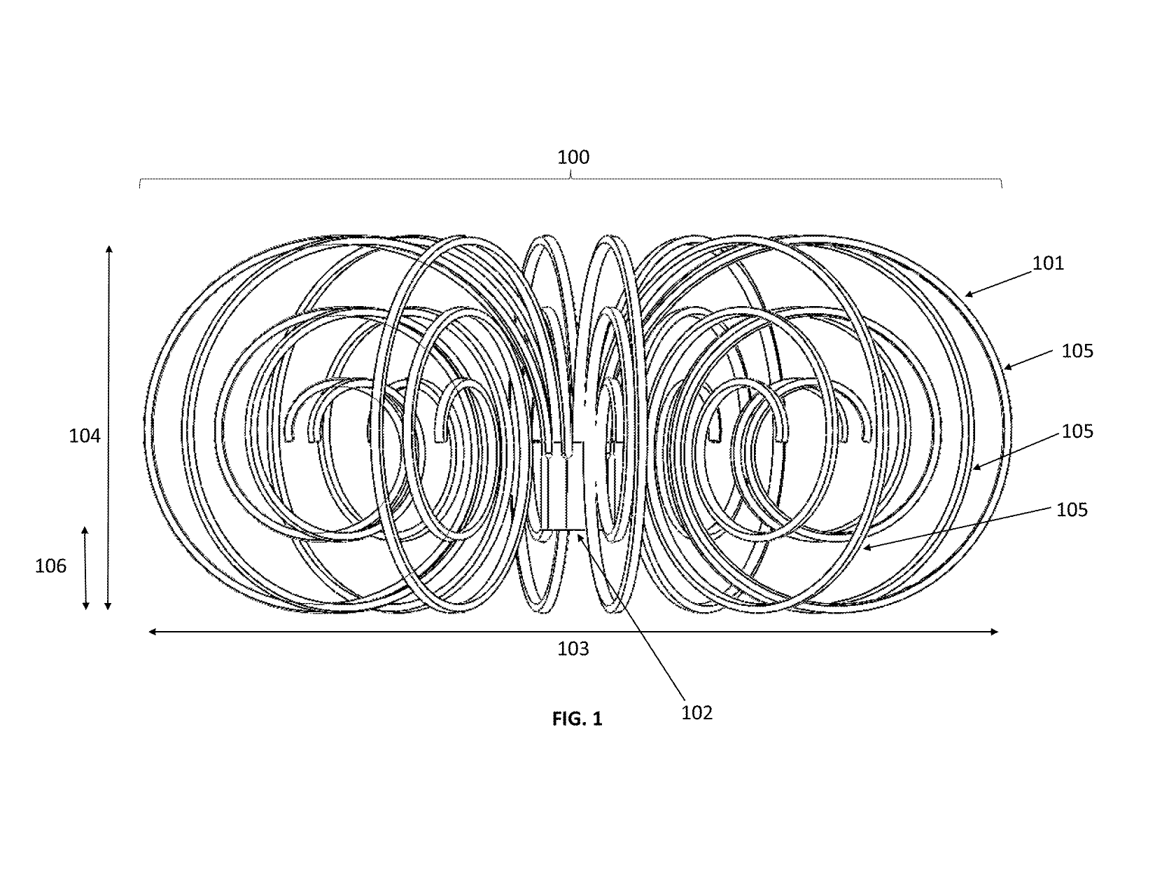

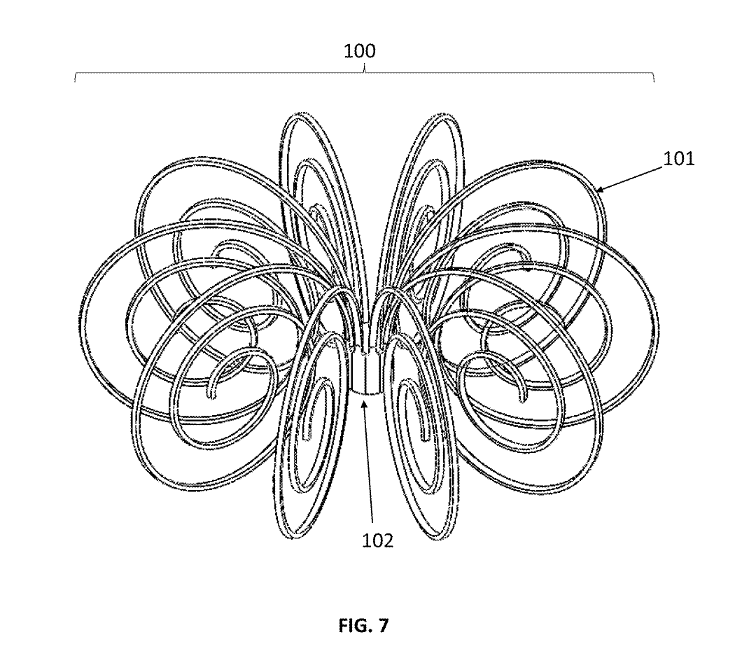

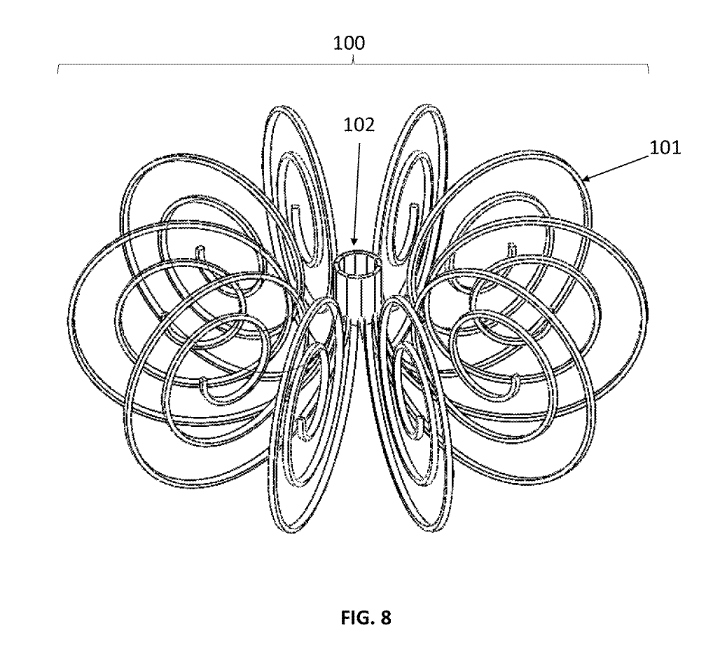

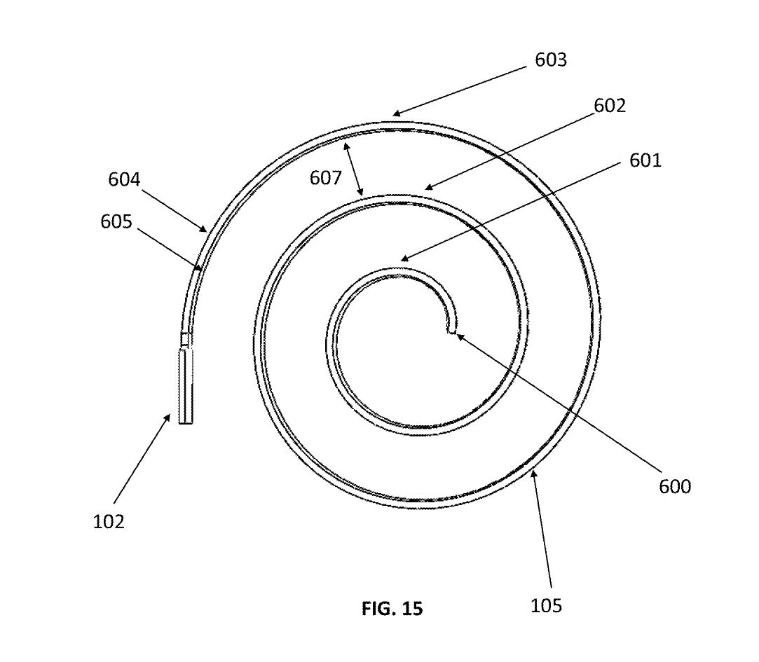

FIG. 1 depicts a deployed left atrial appendage device (LAAD) 100. The LAAD 100 is comprised of several looping struts 105 conjoined at a proximal hub 102. The looping struts 105 create a plurality of outer loops 101, creating a general circular or torus-shaped device as shown in FIGS. 7 and 8, which depict the top and bottom isometric views of the LAAD 100 respectively. Each individual looping strut 105 forms a substantially spiral shape as shown in FIG. 15. The LAAD 100 may be further defined by the deployed section outer diameter 103 and the deployed section height 104. According to an embodiment of the invention, the proximal hub 102 is recessed within the outer loop 101. In this configuration, the proximal hub recess 106 is sufficient to ensure that the proximal hub 102 does not protrude outside of the profile created by the looping struts 105 to reduce the risk of embolization outside of the intended treatment location. In some embodiments, the proximal hub recess 106 eliminates or substantially reduces the amount of material left inside of the left atrium after the LAAD has been implanted. This reduces the risk of potential blood clot formation and, therefore, improves the overall safety to the patient. In some embodiments, the proximal hub recess 106 may also minimize movement of the implanted device by reducing the forces present on the proximal end of the device, where it may otherwise act as a lever arm during normal blood flow. In an alternative configuration (not illustrated), the proximal hub 102 does not have a proximal hub recess 106 and is instead aligned with the proximal end/bottom of the outer loop 101. In this configuration, similar benefits may be observed as described above with the proximal hub recess 106. In addition, having the proximal hub 102 aligned with the proximal end/bottom of the outer loop 101 may promote embolization of the LAA neck, and improve overall healing and recovery time. In another alternative configuration (not illustrated), the proximal hub 102 protrudes outside of the profile created by the looping struts 105 and beyond the outer loop 101. This configuration may allow for additional components to be appended on the proximal hub 102, such as a proximal base 700.

In some embodiments, the LAAD 100 is formed from a material that exhibits superelasticity and shape memory properties, such as nitinol. According to some embodiments of the invention, the LAAD 100 may be laser cut from solid tubing and formed to provide the shape shown in FIG. 1 or other alternatively depicted and undepicted embodiments. Laser cutting provides several advantages over wire braiding, which is another method of manufacturing common in prior art. Laser cutting offers improved mechanical properties, such as a higher radial force and historically better fatigue resistance. Additionally, laser cutting provides advantages in manufacturing and design, as more complexity in the overall design may be implemented more quickly and without the need for intricate tooling. In some embodiments, the LAAD 100 may be formed from a single piece of laser cut material, in that one solid tube may be laser cut, and then shaped, manipulated, and/or formed into the final configuration as described herein. A single-cut design improves the overall manufacturability of the LAAD, increasing the speed of manufacture as well as reducing the overall amount of waste material generated through the process. Single-cut designs also remove the requirement for appending multiple components together, such as with welding, glue-bonding, stitching, or using collars, all of which introduce risk for breakage and fracture at the site of attachment.

In some alternative embodiments, the LAAD 100 may be formed from a wire braid or mesh of a material with similar superelastic and shape memory properties, such as nitinol. In these embodiments, the number of wires used to create the mesh may be increased in order to improve the overall density of the implant in an effort to further reduce blood flow through the LAA neck. This may provide an alternative path to coatings such as ePTFE, which may be present on laser cut devices.

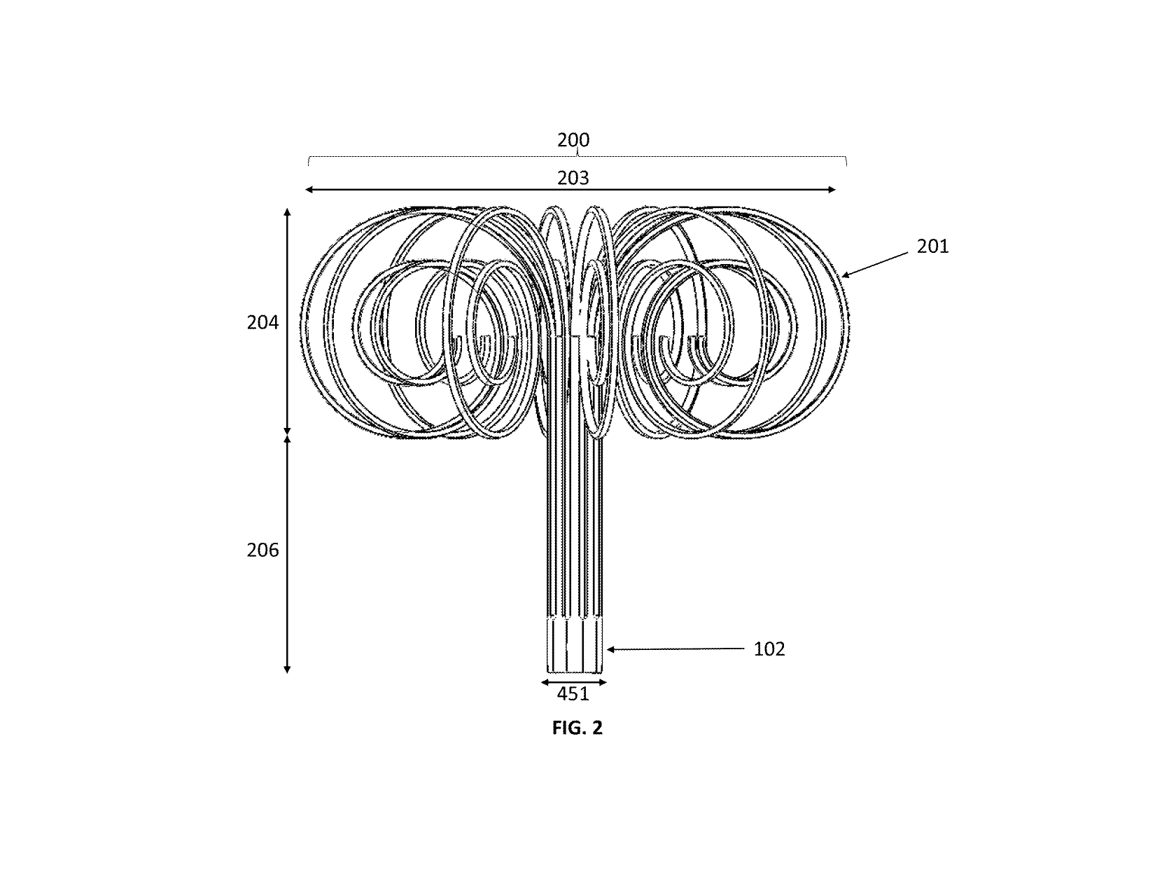

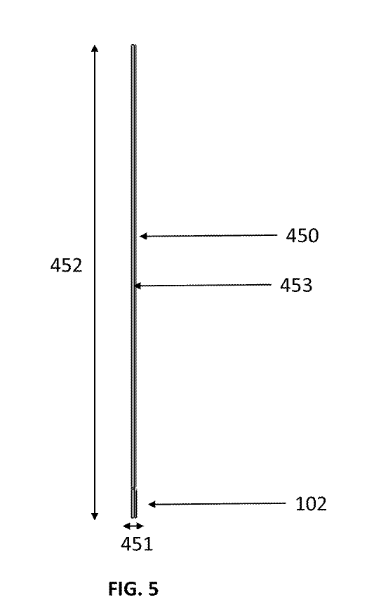

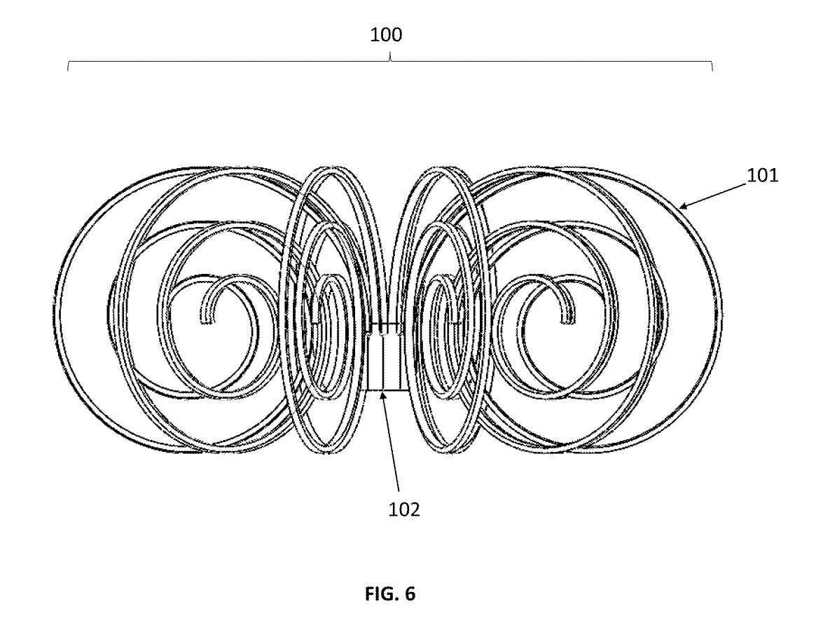

In some embodiments, the LAAD 100 may be advanced through a tube such as a catheter, introducer sheath, or similar loading or introducing mechanism in order to intravascularly reach the intended treatment location. As the LAAD 100 is designed to be advanced intravascularly, the LAAD 100 should preferably have an undeployed outer diameter 451 compatible with the loading or introducing mechanism. In some embodiments, the LAAD 100 may have a predetermined or predefined minimum deployed section outer diameter 103 to be effective and maintain its position against the vessel wall. In some embodiments, the LAAD 100 may have different configurations based upon the stage of deployment that has occurred. In some embodiments, as the LAAD 100 is incrementally unconstrained or unsheathed, the device may begin to roll in upon itself, transitioning from the undeployed LAAD 100 state (as illustrated in FIG. 5) to the deployed LAAD 100 state (as illustrated in FIGS. 1 and 2).

FIG. 5 illustrates the LAAD 100 in an undeployed state, according to some embodiments. In the undeployed state, the LAAD 100 may be substantially tubular and may include a proximal hub 102 and a series of undeployed struts 105 as shown in FIG. 5. In some embodiments, in the undeployed configuration, the undeployed struts 105 may be substantially straight or conform to the geometry of the current tube constraining the device (i.e. loading or introducing mechanism), but may not exhibit any of the looping geometry observed in the struts 105 in the deployed or partially deployed states shown in FIGS. 1-4. In the undeployed state, the LAAD 100 may have an undeployed height 452 that is substantially greater than that of the section height 104 in the deployed state (shown in FIG. 1). In some embodiments, the height 452 of the LAAD 100 in the undeployed state may be proportional to the path length of the looping strut 105 as it connects to the proximal hub 102.

In some embodiments, during advancement and deployment, the LAAD 100 may be progressively unsheathed and, therefore, become unconstrained. In some embodiments, the rolling/looping action of the LAAD 100, shown in FIGS. 1-4, happens continuously throughout deployment and may be characterized by the struts 105 continuously rolling in on themselves in a spiral pattern, thereby forming the loops. The stages described in FIGS. 1-5 are individual snapshots of the deployment process at different points in the process, and do not represent all possible partial deployment configurations that occur during deployment of the LAAD 100.

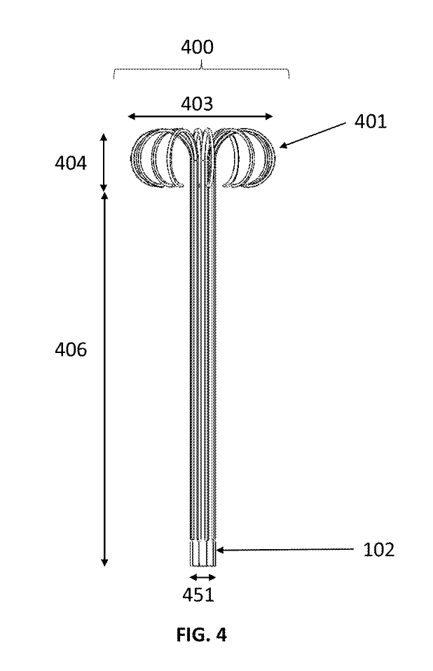

FIG. 4 illustrates a first stage of the deployment process from the undeployed state illustrated in FIG. 5. In some embodiments, the partially deployed LAAD 100 depicted in FIG. 4 may be substantially similar to the undeployed LAAD 100 in FIG. 5 except for the distal end of the LAAD 100, which begins to roll in on itself as it is unsheathed. In some embodiments, the partially deployed LAAD 100 in the first stage of deployment has a single loop 401 with a defined deployed section outer diameter 403 and a partially deployed section height 404. In some embodiments, the deployed section outer diameter 403 is substantially larger than undeployed section outer diameter 451, but less than or equal to the final deployed section outer diameter 103. In some embodiments, the partially deployed LAAD 100 has a partially undeployed section height 406 which is less than the undeployed height 452.

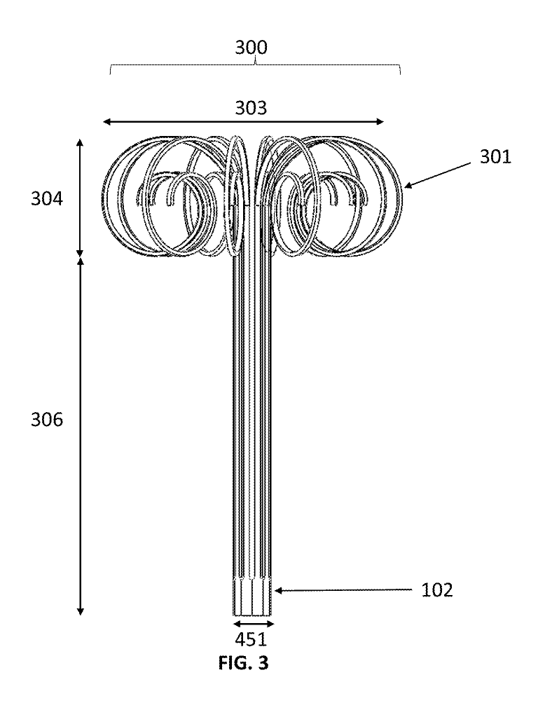

As the deployment process continues, the LAAD 100 progresses from the first stage partial deployment shown in FIG. 4 to a second stage partial deployment shown in FIG. 3. In the second stage of deployment, the partially deployed LAAD 100 has a partially undeployed section with a section height 306 on the proximal end, and a further deployed outer loop shape 301 on the distal end. At this second stage of the deployment, more length of the looping struts 105 has been unsheathed and the struts 105 have, therefore, rolled in on themselves more. In the second stage of partial deployment shown in FIG. 3, the deployed section outer diameter 303 is greater than or equal to the deployed section outer diameter 403 during the first stage of partial deployment (FIG. 4), and substantially greater than the undeployed outer diameter 451. According to some embodiments, the deployed section height 304 during the second stage of deployment (FIG. 3) is substantially similar or equal to the deployed section height 404 during the first stage of deployment (FIG. 4).

The deployment process continues from the second stage shown in FIG. 3 to the third stage of partial deployment of the LAAD 100 as illustrated in FIG. 2. In the third stage of partial deployment, the LAAD 100 is similarly comprised of an undeployed section with a partially undeployed section height 206 on the proximal end, and a series of outer loops with shape 201 on the distal end. At this third stage of the deployment, even more length of the looping struts 105 has been unsheathed and the struts 105 continue to roll in on themselves, forming multiple layers of looping patterns in the spiral created by the looping struts 105. In the third stage of partial deployment, the deployed section outer diameter 203 is greater than or equal to the deployed section outer diameter 303 shown in FIG. 3, and substantially greater than the undeployed outer diameter 451. In some embodiments, the section height 204 in the third stage of partial deployment is substantially similar or equal to the second stage deployed section height 304 and the third stage deployed section height 404 illustrated in FIGS. 3 and 4, respectively.

In some embodiments, the deployment process of the LAAD 100 is completed when the entire undeployed strut 105 depicted in FIG. 5 is converted into a looping strut 105, spiraling in on itself as shown in the deployed LAAD 100 in FIG. 1. In some embodiments, once the LAAD 100 has reached full deployment, an interventionalist may verify the fit of the LAAD 100 at the target location and subsequently detach the LAAD 100 from a corresponding delivery system. In some embodiments, the delivery system may include elements such as a push wire, detachment mechanism, and/or a handle. In some embodiments, the delivery system may include elements for recapture of the LAAD 100, in which case the recapture process would occur in reverse, going from the fully deployed LAAD 100 illustrated in FIG. 1 to the third stage partially deployed LAAD 100 shown in FIG. 2, and so on to the undeployed LAAD 100 shown in FIG. 5, via an unrolling process which can occur by pulling the proximal end of the LAAD 100 into a tube, such as a catheter, introducer sheath, or similar tube, the delivery system.

In some embodiments, throughout the deployment process, the LAAD 100 progressively foreshortens as the looping struts 105 roll in on themselves, transferring axial length to curved path length, with the final deployed LAAD 100 consisting of only the deployed section height 104 which is substantially less than the undeployed height 452. This progressive foreshortening ensures that the part of the LAAD 100 which constitutes the most distal area is constantly changing during deployment, specifically that it is moving towards the center of the loop generated by the looping struts 105. The progressive foreshortening action also ensures that the distal end 600 (see, for example, FIG. 15) of the looping strut 105 has minimal to no ability to come into contact with the walls of the LAA. As the distal end 600 of the looping strut 105 is unattached to any other point on the LAAD 100, the distal end 600 poses the highest risk for damaging the walls of the LAA because it may act as a small area to apply a force to, effectively acting as a point load on the walls of the LAA. Since the looping struts 105 are constantly foreshortening and rolling in on themselves, every additional loop of made by the looping struts 105 further insulates the distal end 600 of the looping strut 105 from the walls of the LAA, further minimizing the risk of damage to the patient.

In some embodiments, the deployed LAAD 100 consists of multiple looping struts 105 which are only connected at the proximal hub 102. The looping struts are not interconnected at any other point along the length of the structure, unless by a secondary material, such as graft material, a coating, or a polymer. The single primary connection point ensures that the looping struts 105 are unconstrained and can roll in on themselves to facilitate intravascular access and atraumatic deployment. The lack of interconnection of the looping struts 105 also permits the LAAD 100 to immediately begin forming the final deployed shape when unconstrained from the catheter or similar insertion tube, significantly reducing the amount of distance required between the distal end of the catheter or similar insertion tube and the distal wall of the left atrial appendage. This reduced distance provides the interventionalist with more flexibility in terms of positioning the LAAD 100 without risking traumatic injury or damage to the patient’s anatomy.

Conventional left atrial appendage occlusion devices without a rolling or unrolling mechanism may have to be fully advanced outside of the catheter or insertion tube prior to these conventional devices taking their final shape. In these instances, the design must balance the overall amount of material with the risk to the patient and difficulty to the physician. The larger the design, and the more material present, the longer the undeployed length of the device will be. As such, it will be more likely that the device will contact the walls of the LAA prior to the device taking on its final shape. Because of this, the interventionalist may have to take the full length of the device into account, and partially deploy the device outside of the LAA to minimize the risk of damaging the distal LAA wall, especially in cases where the LAA is shallow.

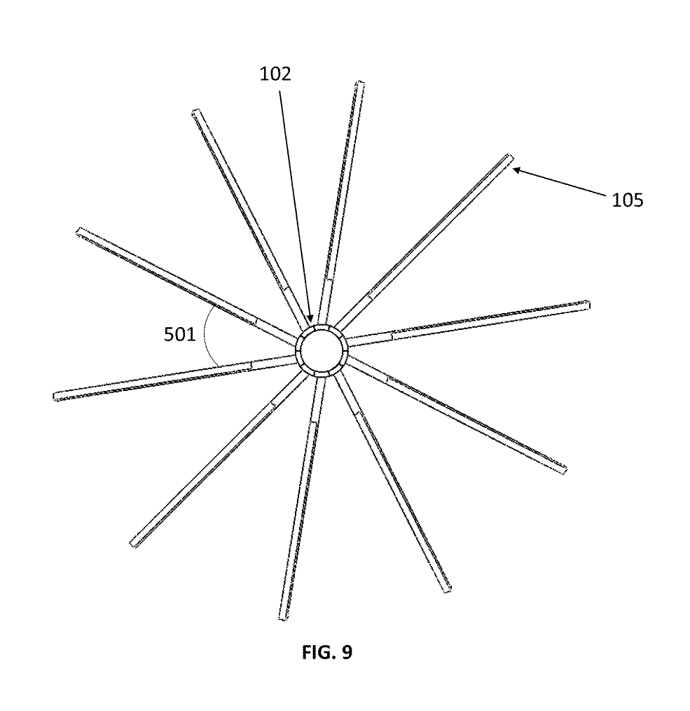

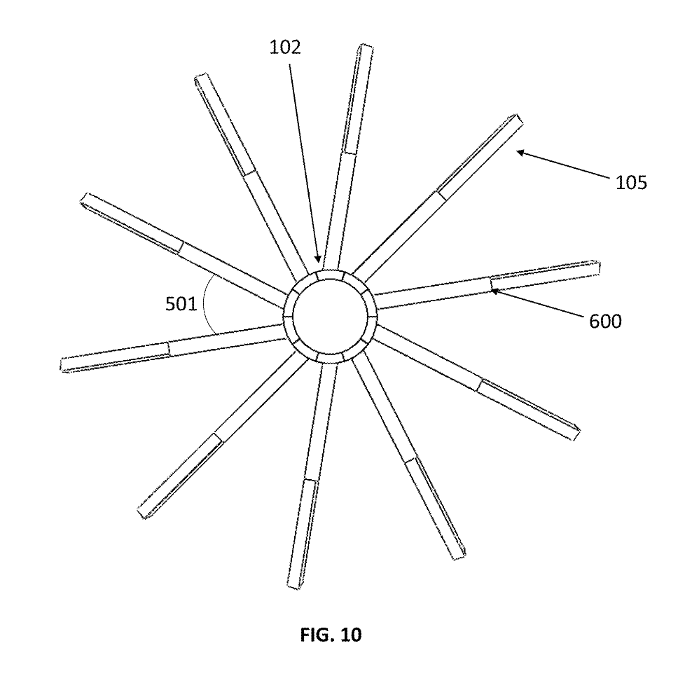

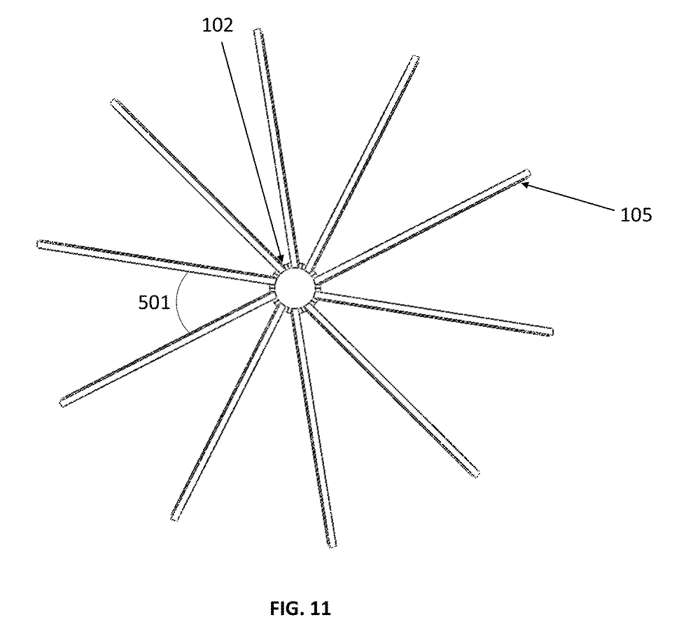

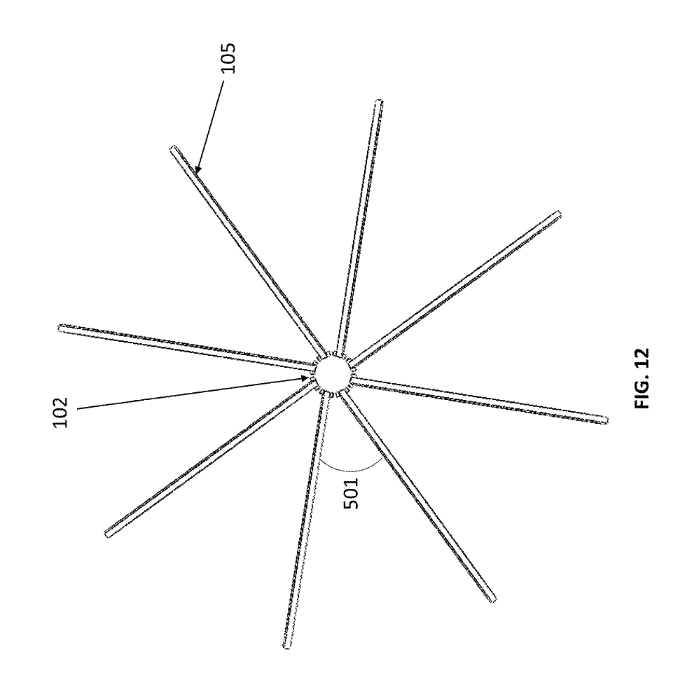

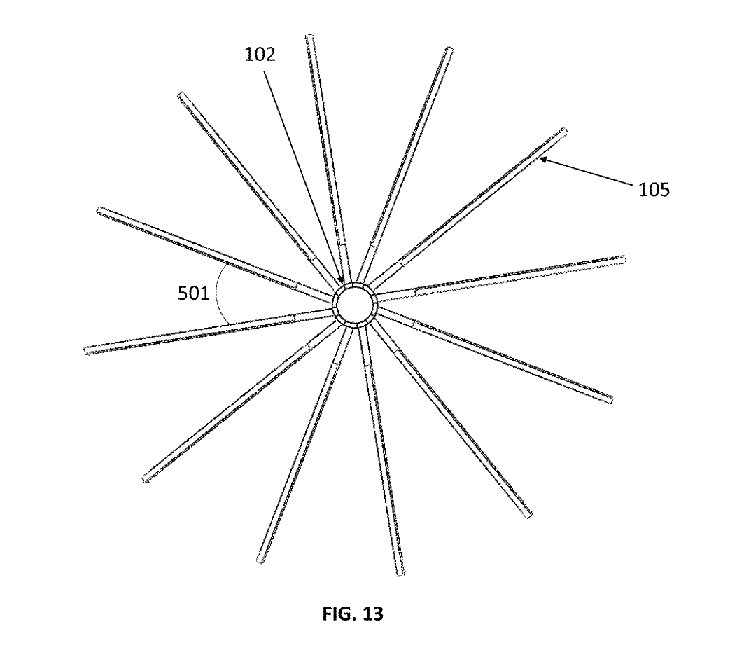

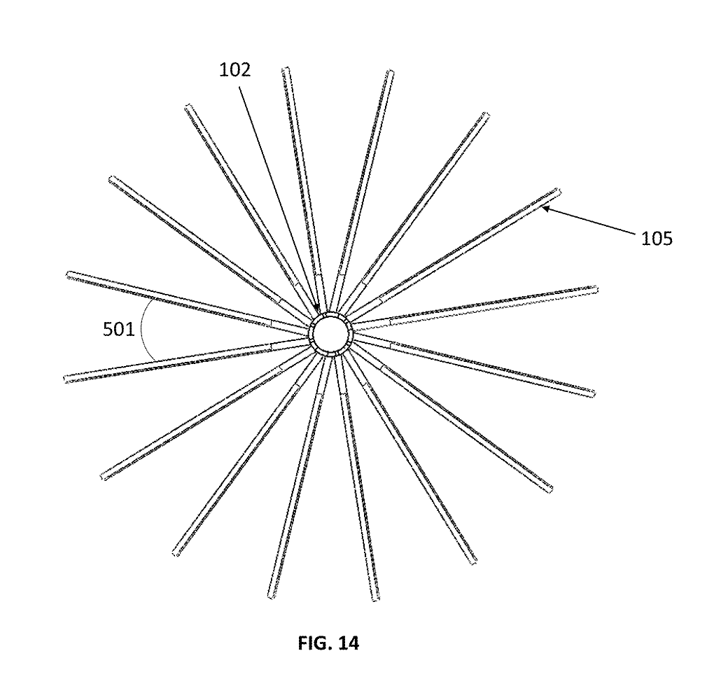

Embodiments of the present invention, with the unrolling mechanism described above, permit the entirety of the device (LAAD 100) to be deployed within the LAA regardless of the depth or size of the LAA, greatly improving patient safety and ease-of-insertion. As the LAAD 100 is advanced out of the catheter or introducer tube, the looping struts 105 immediately begin to roll back on themselves, ensuring that there is minimal distance between the distal end of the catheter or insertion tube and the most distal end of the device at any given moment. Additionally, as the LAAD 100 is inserted into the LAA, the rolling mechanism provides a rounded, atraumatic surface which, even if the strut 105 contacts the LAA wall, would provide a larger area to disperse any applied forces, further minimizing the risk of damaging the LAA wall. This rolling action continues until the entirety of the LAAD 100 has been inserted and deployed in the LAA. As the rolling action is continuous, substantially more material may be included in the implant (LAAD 100) without requiring the interventionalist to deploy the LAAD 100 partially outside of the LAA. In some embodiments, the additional material introduced by the longer length of the looping struts 105 may improve resistance to blood flow to facilitate closure of the LAA. In some embodiments, the deployed LAAD 100 may have a strut-to-strut angle 501 corresponding to the number of looping struts 105 in the design of the LAAD 100. FIGS. 9-14 illustrate various embodiments of the LAAD 100, demonstrating a variable number of looping struts 105. In some embodiments, the minimum number of looping struts 105 is three, and there is no maximum number of looping struts 105, as this is naturally constrained by the looping strut 105 thickness. In some embodiments, the strut-to-strut angle 501 is substantially equivalent between each pair of looping struts 105.

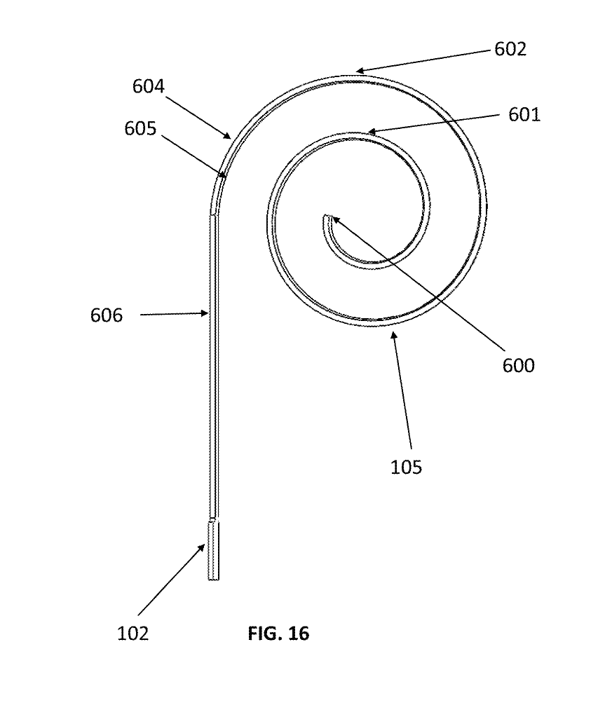

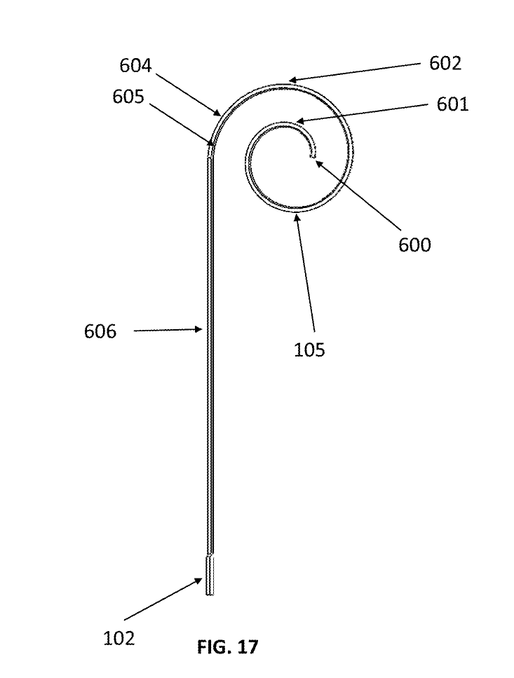

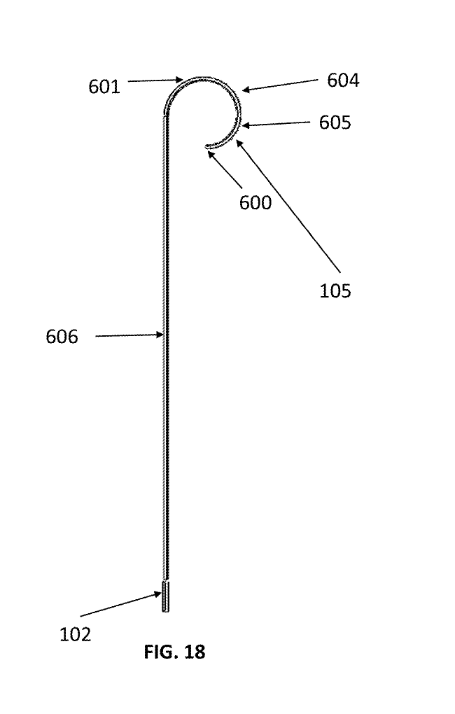

FIGS. 15-18 illustrate a single looping strut 105 during different stages of deployment as previously described with reference to FIGS. 1-5. In some embodiments, the looping strut 105 has a distal end 600 which travels along the radius of the first loop 601, and then radially spirals inward towards the center of the circle inscribed by the first loop 601. Prior to spiraling inward, the undeployed portion 606 of the strut remains straight and constrained within the delivery catheter or sheath. As the device is advanced out of the delivery catheter or sheath the undeployed portion 606 of the strut begins to radially spiral inward, following the spiral initiated by the looping strut 105. In some embodiments, the circles created by the first loop 601, the second loop 602, and the third loop 603 are concentric.

In some embodiments, the rolling action that occurs during deployment of the LAAD 100 involves the strut exterior edge 605 inverting to the interior of the first loop 601 and the strut interior edge 604 inverting to the exterior of the first loop 601. In some embodiments, the strut exterior edge 605 corresponds to the outer surface of the undeployed LAAD 100 (FIG. 5). In some embodiments, the strut interior edge 604 corresponds to the interior surface of the undeployed LAAD 100. This inversion continues through all subsequent loops formed during deployment of the LAAD 100. In some embodiments, this strut inversion may permit usage of sensitive or delicate coatings or surface finishes on the strut interior edge 604. While the strut exterior edge 605 may make contact with and move through a catheter or insertion tube, the strut interior edge 604 may remain undamaged and uncontacted with any surface of the delivery system during the delivery of the LAAD 100 into the LAA. Once deployed, the strut interior edge 604 becomes the surface facing the LAA and, therefore, may benefit from inclusion of surface coatings or similar features.

In some embodiments, the inversion of the looping strut 105 surfaces 604, 605 during deployment improves the ability of the LAAD 100 to be deployed, as the distal end 600 of the looping strut 105 continuously moves inward through the first loop 601 to the second loop 602, and to the third loop 603 or any additional loops beyond that. The distal end 600 of the looping strut 105 is always the closest point to the center of the circles generated by the loops 601-603. Deployment in this fashion also heavily minimizes or completely eliminates the risk of causing trauma at points distal to the deployment site. Because the distal end 600 of the looping strut 105 is always moving inward and away from the distal end of the deployment site, the risk of causing injury to the surrounding tissue is virtually non-existent, even with the distal end 600 being open and not interconnected with other elements of the LAAD 100. This contrasts with prior art, where the distal end of the design may contact the vessel wall during deployment, causing injury to the patient.

In some embodiments, the inversion of the surfaces 604,605 of the looping strut 105 during delivery and deployment ensures that the surface exposed to the catheter or similar introducer tube is different than the surface exposed to the patient's anatomy.

In some embodiments, as the looping strut 105 is further deployed and the spiral shape increases in density, the radial strength of the LAAD 100 increases, further increasing device retention and preventing migration post deployment. The more loops present within the deployed LAAD 100, the higher the radial strength of the LAAD 100 will be. As the spiral shape of the LAAD 100 increases in density, the amount of material present within the neck and proximal end of the LAA increases. As the amount of material in the LAA region increases, the overall resistance to blood flow increases as well. With a sufficient number of looping struts 105, the LAA may close off completely, with blood flow between the LAA and the left atrium minimizing and eventually stopping, resulting in embolization of the LAA and an overall reduction in thrombus risk for the patient with time. Additionally, the increased density of the LAAD 100, in conjunction with any graft materials or coatings, may provide a strong filtering effect prior to embolization of the LAA, where any potential blood clots that may have been generated in the LAA will be trapped within the LAA behind the LAAD 100, and therefore unable to pass through into the left atrium and further downstream where they may pose health risks to the patient.

In some embodiments, the spiral pattern formed by the looping struts 105 has a looping pitch 607, as shown in FIG. 15. In some embodiments, the looping pitch 607 may be increased to reduce the overall number of loops or to increase the deployed section outer diameter 103. In some embodiments, the looping pitch 607 may be decreased to increase the overall number of loops to increase the amount of material present or to decrease the deployed section outer diameter 103. In some embodiments, additional material in the LAAD 100 may be beneficial in providing resistance to blood flow, further promoting embolization and closure of the left atrial appendage. In some embodiments, reducing the amount of material in the LAAD 100 may be beneficial in maximizing deliverability and minimizing device foreshortening. In various embodiments, different deployed section outer diameters 103 and deployed section heights 104 may be required to adequately treat a wide range of patient anatomies. The deployed section outer diameter 103 may be increased in some embodiments to accommodate an LAA with a wide neck or opening or to accommodate an overall larger LAA. In various embodiments, due to the nature of the rolling action of delivery, different size LAADs 100 may be able to use the same or similar size catheter or introducer tube sizes for advancement and delivery. While the presence of additional material in the deployed state may correspond to a longer LAAD 100 in the undeployed state, a larger diameter catheter or introducer tube is not required to accommodate the additional material.

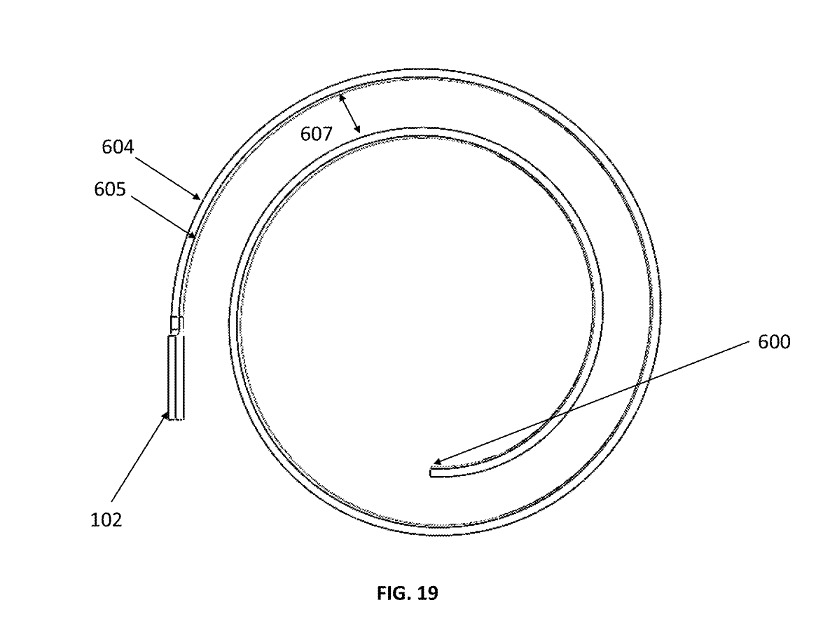

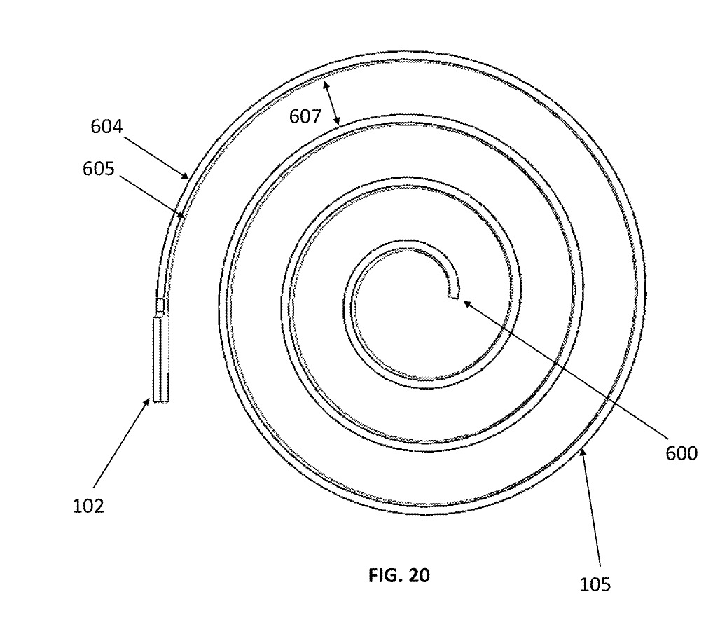

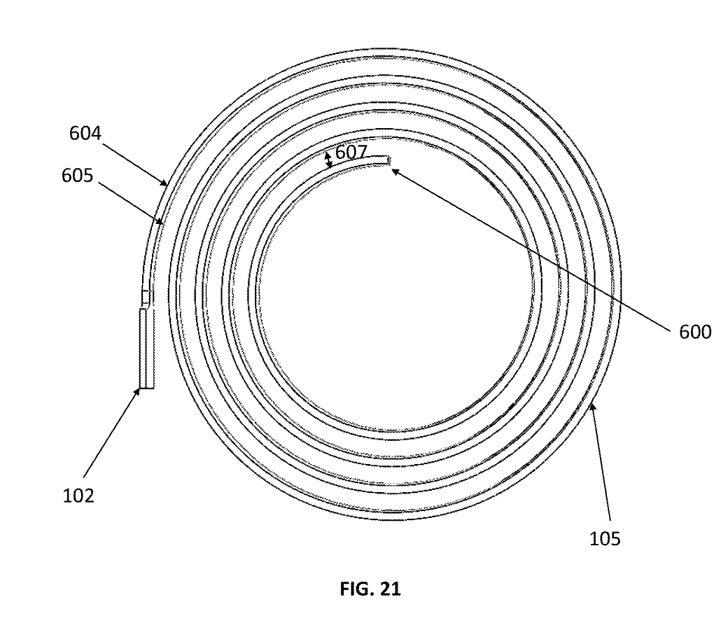

Various alternative embodiments of the spiral pattern are depicted in FIGS. 19-21 and illustrate a variable number of loops and looping pitches 607. In some embodiments, variations in the spiral pattern and number of loops and looping pitches may increase radial strength and resistance to blood flow, further improving efficacy of the LAAD 100. The number of loops must be at least one but is otherwise only constrained by the size of the LAA and the thickness of the device (LAAD 100) material.

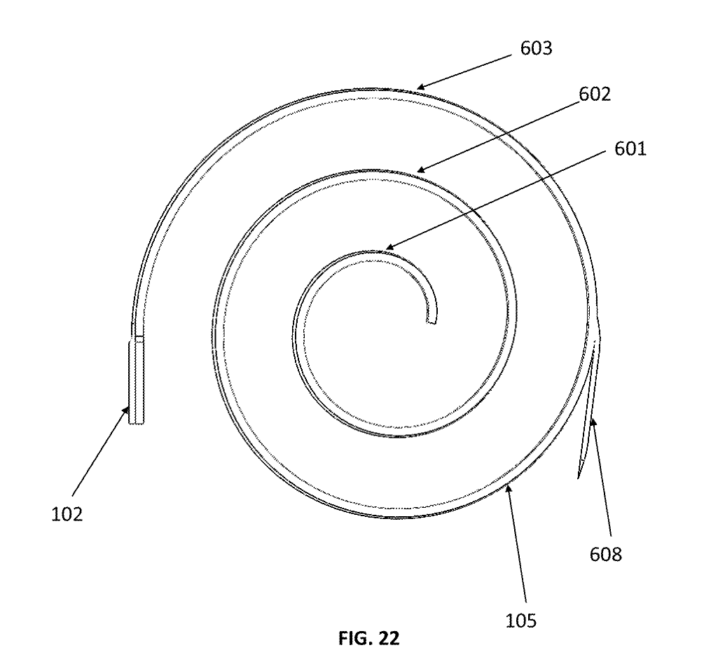

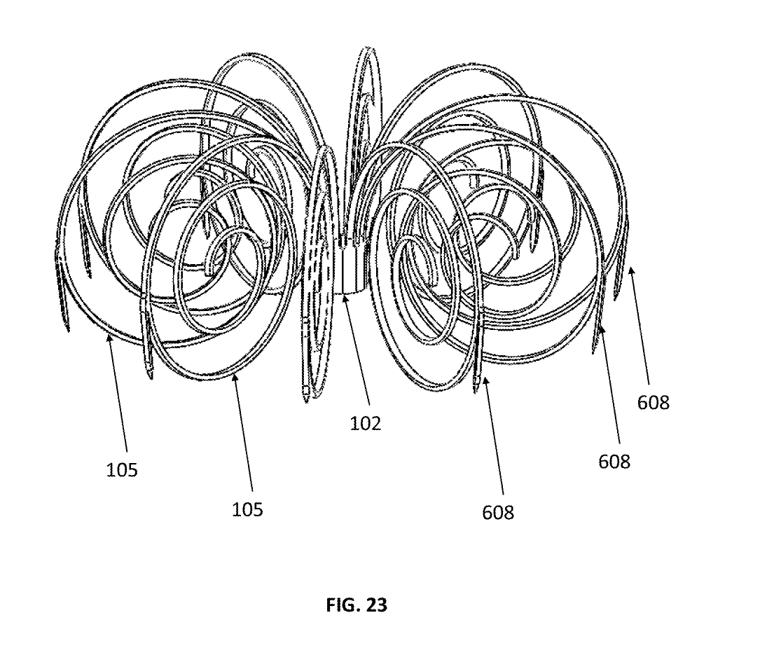

In some embodiments, the LAAD 100 may include barbs or protrusions 608 along the length of the looping struts 105, as illustrated in FIGS. 22-26, to improve device retention and decrease the risk of migration after implantation. The barbs or protrusions 608 may be positioned on the looping struts 105 to ensure that they are positioned on the outer loop 101 (corresponding to the third loop 603). In some embodiments, during deployment, as the looping strut 105 rolls, the barbs or protrusions 608 may further advance into surrounding tissue, increasing retention. Should the LAAD 100 have to be recaptured or removed, the barbs or protrusions 608 are removed in the same direction as they were inserted, minimizing the potential trauma to surrounding tissue. In some embodiments, during recapture of the LAAD 100, the looping struts 105 will begin to roll back (unroll) into the catheter or introducer tube following the same path that they followed during insertion. As such, the barbs or protrusions 608 will be removed in a similar fashion as to how they were inserted, providing minimal risk of tearing or cutting the tissue during recapture. Alternatively, in some embodiments, the LAAD 100 may be deployed inside of the LAA and subsequently pulled proximally to engage the barbs or protrusions 608 to the LAA wall. An example of the LAAD 100 containing barbs or protrusions 608 is illustrated in FIGS. 22-23. In alternative embodiments of the invention, the barbs or protrusions 608 may be present at any point along the length of the third loop 603, or the most exterior loop in the case where there are more or less than three loops in the design.

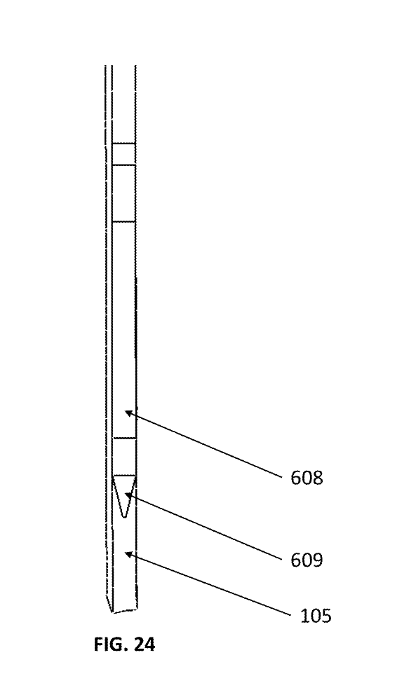

FIG. 24 illustrates the barb or protrusion 608 relative to a strut 105, according to some embodiments. The barb or protrusion 608 has a barb tip 609. The barb tip 609 has a pointed end, to facilitate puncture into the wall of the LAA. The barb tip 609 enters the LAA wall to provide additional securement of the LAAD 100 to the LAA and minimize the risk of movement or migration of the LAAD 100 after implantation. In some embodiments, the barb or protrusion 608 has a width similar to the width of the looping strut 105. In these embodiments, the barb or protrusion 608 may be adhered onto the surface via a secondary process such as welding, stitching, or glue bonding.

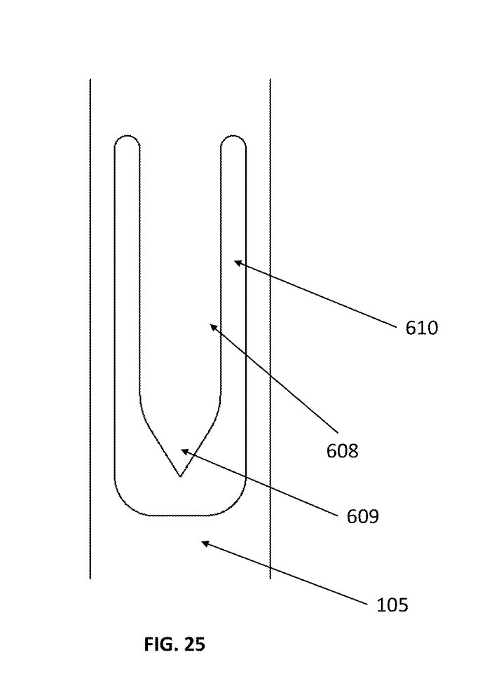

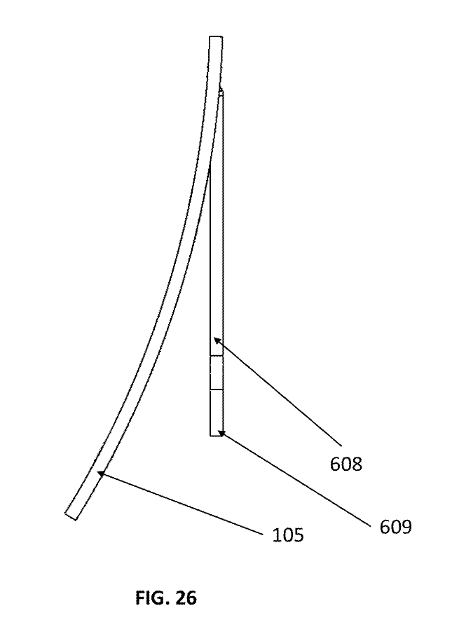

In some embodiments, as shown in FIGS. 25 and 26, the barb or protrusion 608 may be cut out of the looping strut 105 such that it is formed from the same material in a single laser cut geometry. In these embodiments, there is minimal risk of fracture at the joint or connection point, as the material is continuous through the looping strut 105 and the barbs or protrusions 608. In these embodiments, the barb or protrusion 608 has a width less than that of the looping strut 105. The size of the barb cutout 610 may be as narrow as one kerf width of a laser beam used for the laser cutting, which maximizes the widths of both the looping strut 105 and the barb or protrusion 808. Alternatively, the barb cutout 610 may be larger, with gap distances up to one quarter of the width of the looping strut 105, which may provide easier manufacturing and shape setting of the LAAD 100.

In some embodiments, the barbs or protrusions 608 do not follow the same arcuate shape or path as the looping struts 105 and, instead, remain tangent to the arc of the looping struts 105, as shown in FIG. 26. In some embodiments, the barbs or protrusions 608 may be positioned such that the barb tip 609 is oriented downward towards the proximal end of the LAAD 100 so that the barb tip 609 can engage the LAA wall during deployment of the LAAD 100.

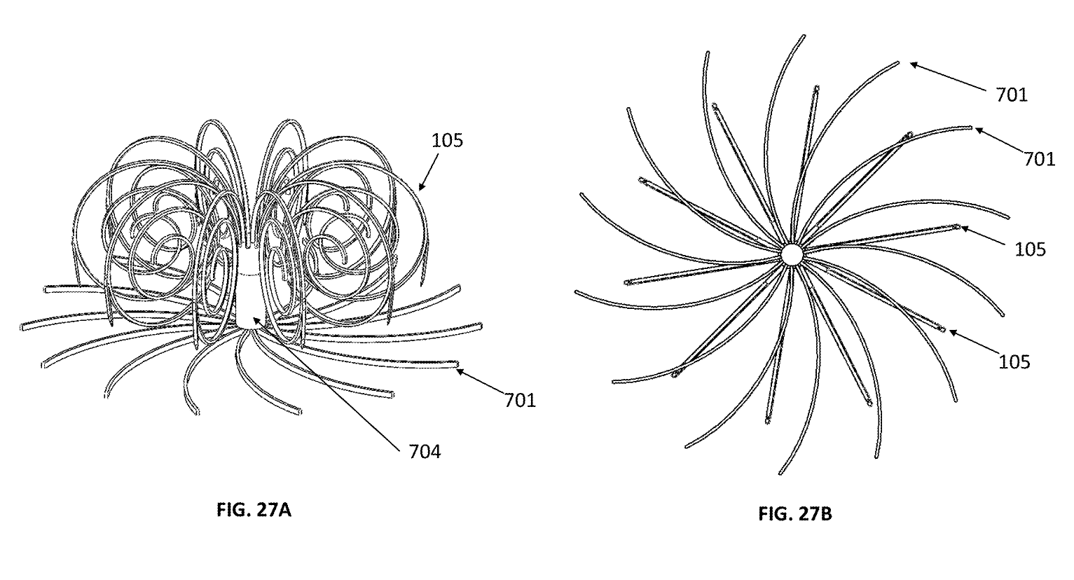

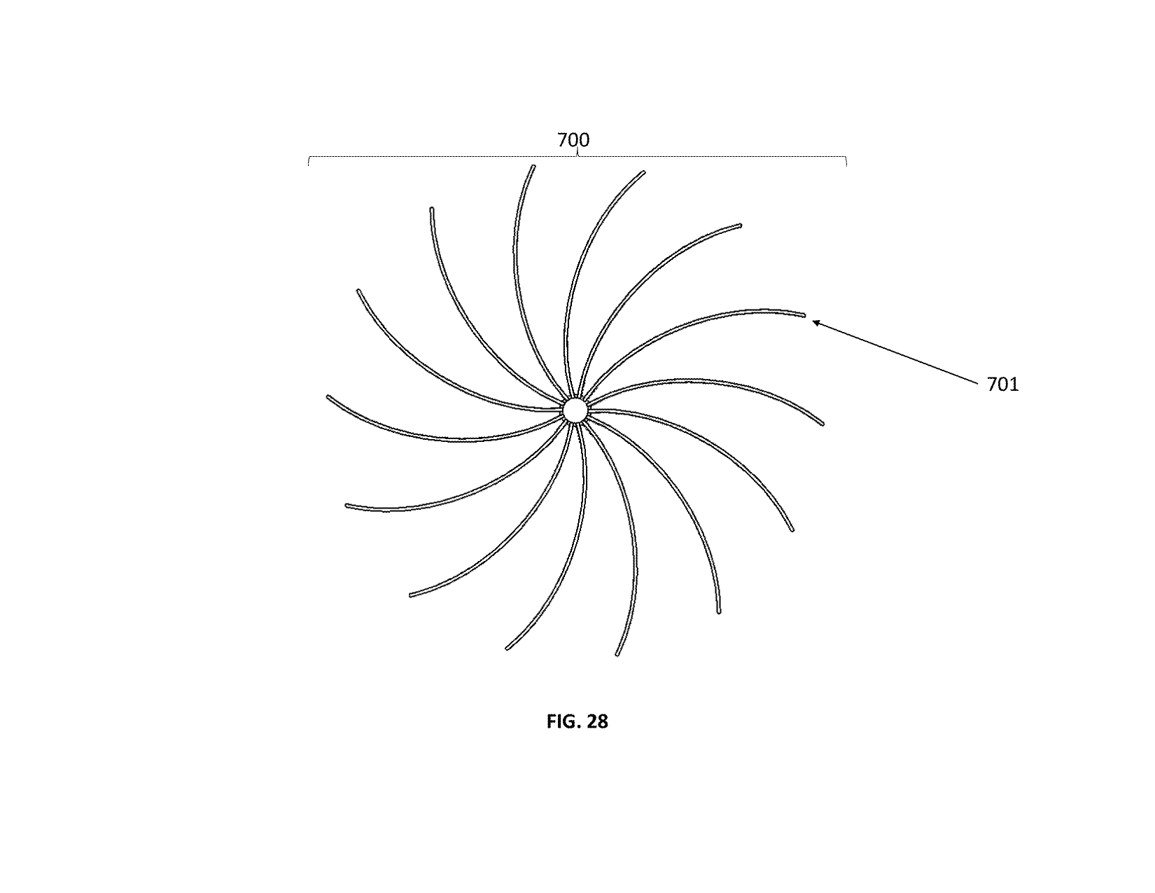

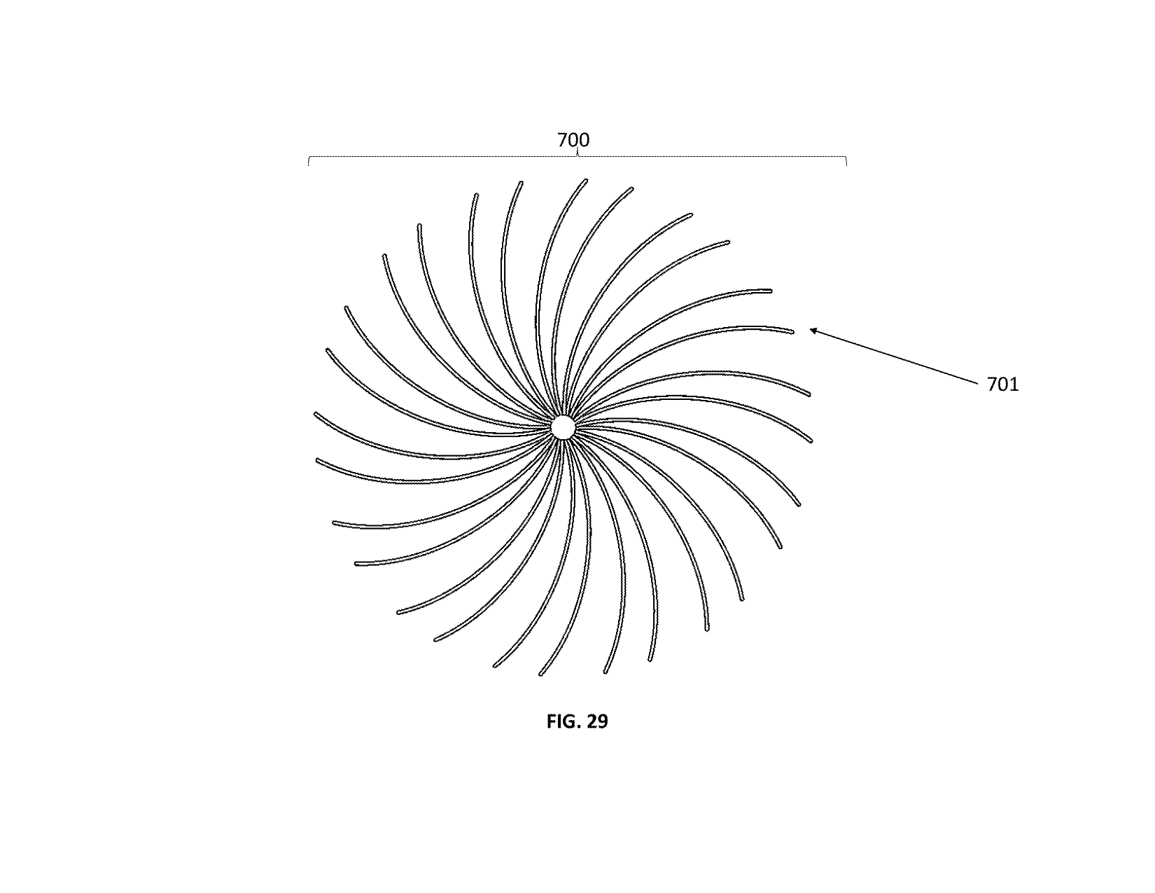

In some embodiments, the LAAD 100 may include a proximal base 700 which is comprised of a plurality of base struts 701 and a connector 704, as illustrated in FIGS. 27-31. In some embodiments, the connector 704 attaches the proximal base 700 to the proximal hub 102, either through a single piece construction where the proximal base 700, the connector 704, and the proximal hub 102 are formed as a single element or via an attachment method such as welding, glueing, epoxying, or stitching. In some embodiments, the proximal base struts 701 have a defined strut width and a strut thickness, and form a substantially circular pattern. In some embodiments, the proximal base 700 further improves retention and prevents migration of the LAAD 100 after implantation. In some embodiments, in the deployed configuration, the circular or torus shaped portion 705 of the LAAD 100 resides entirely within the LAA, while the proximal base 700 of the LAAD 100 resides outside of the LAA, for example, inside of the left atrium. In some embodiments, a space or gap between the proximal base 700 and the circular or torus shaped portion 705 is occupied by the connector 704, which is large enough to provide a gap for the neck of the LAA to reside within. The neck of the LAA is then trapped between the proximal base 700 and the circular or torus shaped portion 705, preventing migration or movement of the LAAD 100 post implantation. In some embodiments, the height of the connector 704 may be reduced or minimized to cause the LAAD 100 to compress on the LAA neck, thereby further minimizing the risk of migration of the LAAD 100 post implantation.

In some embodiments, the proximal base 700 may be formed of a plurality of base struts 701 arranged in a circular pattern. The base struts 701 may be curved, straight, or comprised of alternative patterns such as waves or zig-zags. In some embodiments, the base struts 701 are not interconnected on the outer ends, such that each base strut 701 is only connected to the connector 704 as shown in FIG. 27A. In some embodiments, the base struts 701 may be curved distally so that they exert a force into the left atrial wall to further improve device retention.

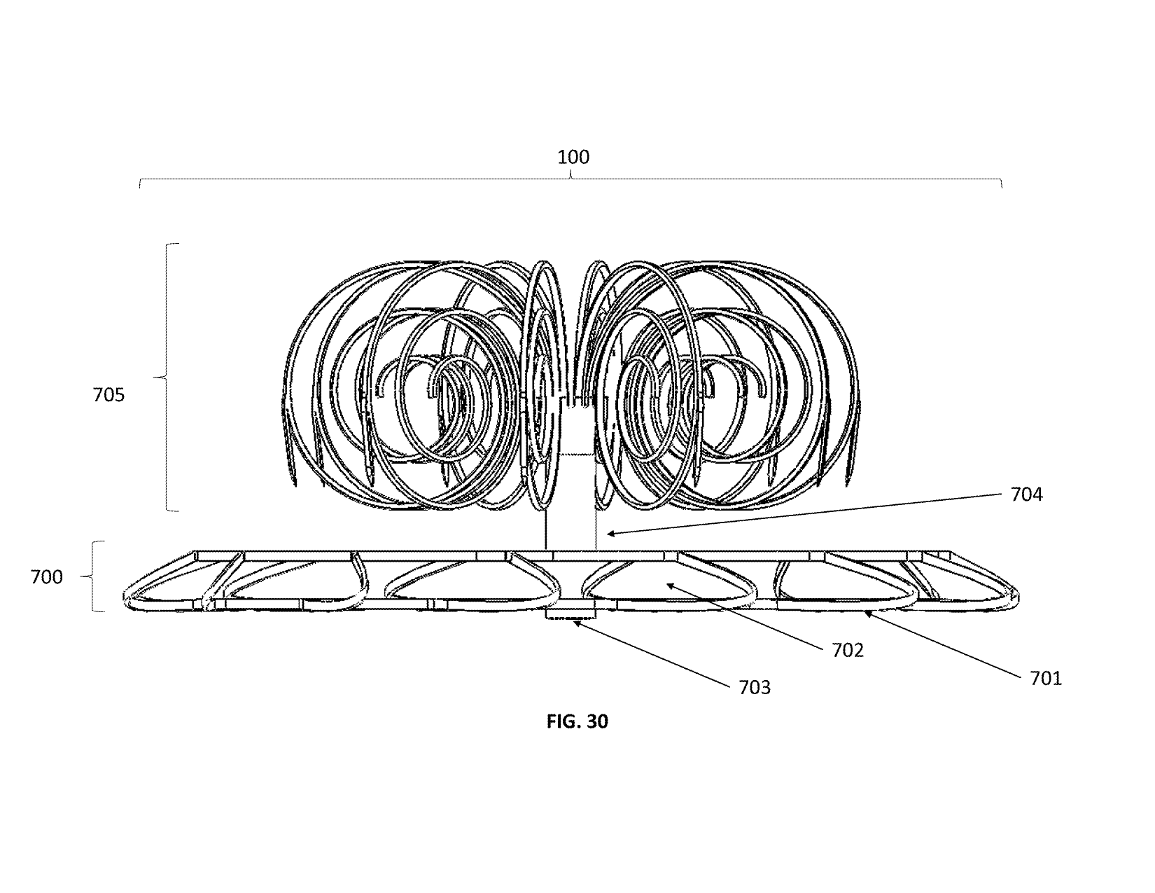

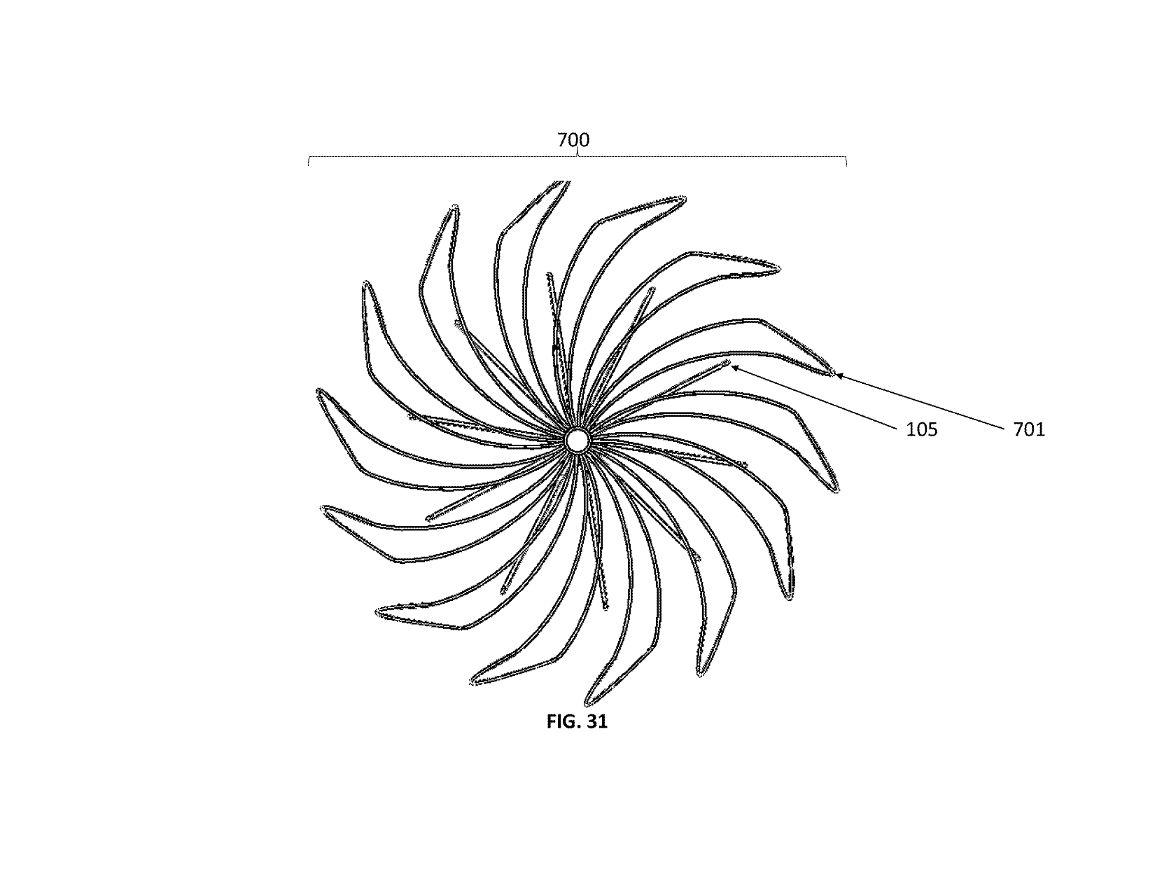

In some embodiments, the base struts 701 form a looping pattern such that they are connected at the connector 704 and at a base hub 703, as shown in FIGS. 30 and 31. In these embodiments, the looping pattern of the base struts 701 forms a base cavity 702. In some embodiments, filter material, graft material, or similar void-filling materials may be incorporated into the base cavity 702 to further increase the resistance to blood flow and to provide additional filtering effects to improve the efficacy of the LAAD 100. In some embodiments, the base hub 703 is located within the base cavity 702 to minimize the risk of protruding material within the left atrium. In some embodiments, the base hub 703 may be in-line with the proximal or bottom end of the proximal base 700 to provide a smooth surface to promote consistent healing. In some embodiments, the base hub 703 may protrude outside of the proximal or bottom end of the proximal base 700 for ease of manufacturing or to allow for recapture or repositioning of the LAAD 100 post deployment. The height of the base cavity 702 is dependent upon the arc of the base struts 701 as they loop around, and is ideally minimized to create the lowest profile within the left atrium.

In some embodiments, barbs or protrusions similar to those on the circular or torus shaped portion 705 may be incorporated onto the base struts 701 to further improve device retention and minimize the risk of migration of the LAAD 100 post implantation.

In some embodiments\, the LAAD 100 may be coated with additional materials such as graft material, electrospun fibers, stitched polymers, or similar materials to facilitate left atrial appendage closure. In some embodiments, the graft material may be connected throughout the length of the looping strut 105, extending from the proximal hub 102 to the distal end of the loop 600. In some embodiments, the graft material may be connected to only a portion of the LAAD 100, such as only the proximal portion of the looping strut which makes up the outer loop 101. In some embodiments. the height of the graft material may correspond to the path length created by the outer loop 101 to ensure that the outer loop 101 is fully covered in graft material. In some embodiments, the graft material may be positioned to be between the patient tissue and the looping strut 105 when fully deployed. In some embodiments, the graft material may provide a filtering action by capturing larger particles and blood clots within the LAA and preventing them from exiting the LAA and into the larger blood stream. In some embodiments, the graft material may also provide additional resistance to blood flow, further promoting embolization of the LAA.

In some embodiments, the LAAD 100 may include radiopaque elements, such as alternative materials, coatings, collars, coils, or other elements appended on the distal or proximal ends of the LAAD 100 to facilitate visibility by the interventionalist during the procedure.

It should be understood that the invention is not limited to the embodiments discussed above, which are provided for purposes of illustration only. Subsets or combinations of various embodiments described above provide further embodiments of the invention.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. An implantable device for occluding a left atrial appendage, comprising:
a frame including a proximal hub and a plurality of struts interconnected at the proximal hub, the frame configured to be implanted at a neck region of a left atrium,
wherein, in an undeployed configuration, the plurality of struts are substantially linear in shape, and
wherein, in a deployed configuration, the plurality of struts are biased to deform radially inward, such that each strut follows a spiral path forming a plurality of loops, with an outer loop that engages with tissue in the left atrium and a free distal end disposed radially inward at an end of an innermost loop.

2. The implantable device of claim 1, wherein the proximal hub is recessed within a profile defined by the outer loop to reduce exposure of the proximal hub to the left atrium.

3. The implantable device of claim 1, wherein the proximal hub is aligned with a proximal end of the outer loop to promote embolization at a neck region of the left atrium.

4. The implantable device of claim 1, wherein the proximal hub protrudes proximally beyond the outer loop and includes an attachment feature configured to couple to and detach from a delivery mechanism.

5. The implantable device of claim 1, wherein, in the deployed configuration, the plurality of struts collectively form a generally annular or toroidal occluding portion sized to conform to a neck region of the left atrial appendage.

6. The implantable device of claim 1, wherein the plurality of loops formed by each strut are concentric and define a spiral pattern that increases in density radially inward.

7. The implantable device of claim 1, wherein each strut of the plurality of struts inverts during deployment such that an exterior surface of each strut in the undeployed configuration becomes an interior-facing surface in the deployed configuration.

8. The implantable device of claim 1,
wherein each strut includes a barb or protrusion disposed along the outer loop and configured to engage the tissue at a neck region of the left atrium, and
wherein the barb or protrusion remains substantially tangent to an arcuate curvature of the outer loop to facilitate atraumatic recapture of the device.

9. The implantable device of claim 8, wherein the barb or protrusion is formed integrally with each strut by a cutout in the strut.

10. The implantable device of claim 8, wherein the barb or protrusion is affixed to each strut by welding, bonding, stitching, or adhesive.

11. The implantable device of claim 1, wherein the frame comprises a superelastic, shape-memory alloy.

12. The implantable device of claim 1, wherein the frame is laser cut from a single piece of tubing and shape set to bias the plurality of struts to deform into a spiral path during deployment.

13. The implantable device of claim 1, further comprising a membrane spanning between adjacent struts to increase resistance to blood flow through a neck region of the left atrium.

14. The implantable device of claim 13, wherein the membrane covers at least the outer loop to provide a filtering effect that traps thrombus within the left atrial appendage.

15. The implantable device of claim 1, further comprising one or more radiopaque elements disposed at the proximal hub or along at least one strut to facilitate visualization during deployment.

16. The implantable device of claim 1, further comprising a proximal base coupled to the proximal hub by a connector, the proximal base including a plurality of base struts arranged to form a substantially circular support outside the left atrial appendage.

17. The implantable device of claim 16, wherein the connector defines a gap configured to correspond to a neck region of the left atrium such that the neck region is trapped between the proximal base and the occluding portion to resist migration of the device.

18. The implantable device of claim 16, wherein the proximal base defines a base cavity formed by looping base struts, the base cavity configured to receive a filter or graft material to further impede blood flow.

19. The implantable device of claim 1, wherein the device is configured for recapture by proximally withdrawing the device into a delivery mechanism such that the plurality of struts unroll along a path opposite to deployment.

20. The implantable device of claim 1, wherein the device is configured for incremental deployment from a delivery mechanism such that the distal end of each strut progressively moves radially inward as additional length of the strut is unconstrained.
